(19) United States
(12) Patent Application Publication
(10) Pub. No.: US 2020/0354297 A1
(43) Pub. Date: Nov. 12, 2020

(54) Methods of Manufacturing Cannabidiol or Cannabidivarin and Intermediates of Manufacturing Cannabidiol or Cannabidivarin

(71) Applicant: Fresh Cut Development, LLC, Chandler, AZ (US)

(72) Inventors: James Patrick Mitchell, JR., Austin, TX (US); Patrick Prince, Austin, TX (US)

(21) Appl. No.: 16/870,637

(22) Filed: May 8, 2020

Related U.S. Application Data
(60) Provisional application No. 62/846,279, filed on May 10, 2019.

(51) Int. Cl. C07C 37/84; C07C 47/565; C07C 37/74

(52) U.S. Cl. CPC C07C 37/84; C07B 2200/13; C07C 37/74; C07C 47/565

(57) ABSTRACT

Methods of manufacturing cannabidiol (CBD) and cannabidivarin (CBDV); intermediates of the methods of manufacturing CBD and CBDV; and crystallized CBD and CBDV obtained via described methods.

### RELATED APPLICATIONS

This application claims priority to U.S. application No. 62/846,279, filed May 10, 2019 (expired). The entire contents are which are hereby incorporated herein by reference in their entirety.

### FIELD OF THE INVENTION

The present invention relates generally to methods of manufacturing cannabidiol (CBD) or cannabidivarin (CBDV); intermediates used in the methods; and crystallized cannabidiol or cannabidivarin of excellent purity.

### BACKGROUND OF THE INVENTION

Cannabinoids are chemicals that are produced mainly by cannabis flowers. Cannabinoids imitate endogenous compounds in humans.

Cannabinoids (for the purposes of the present invention, a cannabinoid is defined as any compound that is active at the cannabinoid receptors) include cannabinol, cannabidiol, cannabigerol, cannabichromene, cannabicyclol, dronabinol (delta-9-tetrahydrocannabinol), delta-8-tetrahydrocannabinol, 11-hydroxy-tetrahydrocannabinol, 11-hydroxy-delta9-tetrahydrocannabinol, levonantradol, delta-11-tetrahydrocannabinol, tetrahydrocannabinol, tetrahydrocannabinolic acid, cannabivarin, cannabidivarin, cannabichromevarin, cannabigerovarin, cannabigerol monomethyl ether, cannabielsoin, cannabicitran, cannabidiolic acid, tetrahydrocannabivarin, anandamide, nabilone, and acids and analogs thereof. It is now possible to synthesize many cannabinoids in a laboratory thereby eliminating the need to grow cannabis for extraction of the compounds.

One cannabinoid, cannabidiol (CBD), (−)-trans-2-p-mentha-1,8-dien-3-yl-5-pentylresorcinol, is non-psychoactive and has shown promise in treating numerous diseases and disorders. Synthetic cannabidiol has the same structure as naturally occurring cannabidiol. The structure of CBD is reproduced below:

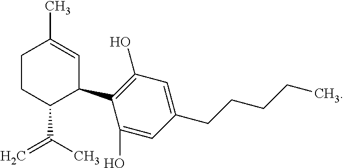 [SMILES: C=C(C)[C@@H]1CCC(C)=C[C@H]1c1c(O)cc(CCCCC)cc1O]

One of the main processes for manufacturing CBD has been described in the seminal paper Petrzilka, T., W. Haefliger, and C. Sikemeier, Synthese von Haschisch-Inhaltsstoffen.4. Mitteilung. Helvetica Chimica Acta, 1969, 52(4), p. 1102-1134.

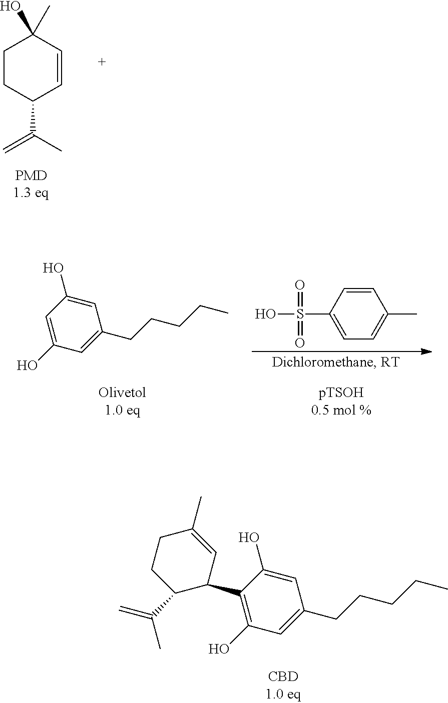

However, there are several disadvantages of using this process. Both of the starting compounds, PMD (a natural product derived from limonene) and olivetol (another natural product that may be derived from olives or lichens), are very reactive compounds. The process, therefore, typically requires using a lot of these compounds for industrial manufacturing. In addition, because of multiple alkylation centers, the reaction produces by-products, such as abnormal-CBD and so-called “bis-adducts.” Furthermore, the process requires labor-intensive chromatographic purification of CBD, resulting in a low overall yield and a time-consuming laborious process.

Accordingly, there is a need in the art for improved methods of manufacturing cannabinoids such as CBD and CBDV.

### SUMMARY OF THE INVENTION

In one embodiment, the invention provides a method of manufacturing cannabidiol (CBD) comprising the following steps:

a) reacting p-mentha-2,8-diene-1-ol (PMD) with 6-carboxymethyl olivetol (CMO) and a catalyst (preferably, boron trifluoride (BF₃)-etherate) in the presence of an organic solvent to produce (1′R,2′R)-methyl 2,6-dihydroxy-5′-methyl-4-pentyl-2′-(prop-1-en-2-yl)-1′,2′,3′,4′-tetrahydro-[1,1′-biphenyl]-3-carboxylate (CMCBD);

b) distilling CMCBD to purify it via thin film evaporation;

c) reacting the distilled CMCBD with water, methanol and sodium hydroxide to produce CBD; and

d) purifying the CBD from step (c) to obtain a purified CBD.

In a preferred embodiment, the purifying of CBD in step (d) is performed via crystallization to obtain a crystallized CBD.

In a preferred embodiment, the molar ratio of PMD to CMO in step (a) is from 1:1 to 10:1.

Preferably, PMD is in at least a 30% molar excess compared to CMO.

In another preferred embodiment, step (a) is carried out at a temperature from 10° C. to 30° C.

In one embodiment, the organic solvent in step 1 is selected from dichloromethane, ethyl acetate, chloroform, methyl tert-butyl ether, cyclohexane, toluene, ethyl alcohol, methyl alcohol, isopropyl alcohol, n-butyl alcohol, tetrahydrofuran, dioxane, dimethylformamide, dimethyl sulfoxide, dimethylacetamide, methyl tert-butyl ether, cyclohexane, water and mixtures thereof.

In another embodiment, the distilled CMCBD obtained in step (b) has a purity of at least 95%.

In another embodiment, step (b) is carried out at a temperature from 70° C. to 170° C.

In one embodiment, the molar ratio of CMCBD and water in step (c) is from 1:1 to 1:100.

In another embodiment, the molar ratio of methanol and sodium hydroxide in step (c) is from 1:1 to 1:100.

In a preferred embodiment, step (d) does not include chromatography purification, and therefore is a non-chromatographic process.

In one embodiment, step (d) comprises using hexane and/or pentane as a crystallization solvent.

In another embodiment, the crystallized CBD obtained in step (d) has a purity of at least 99%.

In one embodiment, the crystallized CBD obtained in step (d) has the following crystal size distribution: between 250 μm and 1000 μm, with average size being 500 μm.

The invention also provides a crystallized CBD manufactured by the method of claim 1.

In one embodiment, the invention provides a method of manufacturing cannabidivarin (CBDV) comprising the following steps:

a) reacting p-mentha-2,8-diene-1-ol (PMD) with 6-carboxymethyl divinarol (CMD) and a catalyst in the presence of an organic solvent to produce (1′R,2′R)-methyl 2,6-dihydroxy-5′-methyl-4-propyl-2′-(prop-1-en-2-yl)-1′,2′, 3′,4′-tetrahydro-[1, 1′-biphenyl]-3-carboxylate (CMCBDV);

b) distilling CMCBDV to obtain a distilled CMCBDV;

c) reacting the distilled CMCBDV with water, methanol and sodium hydroxide to produce a CBDV; and

d) purifying the CBDV from step (c) to obtain a purified CBDV.

In an embodiment, the purifying of CBDV in step (d) is performed via crystallization to obtain a crystallized CBDV.
The invention also provides a compound of the following structures:

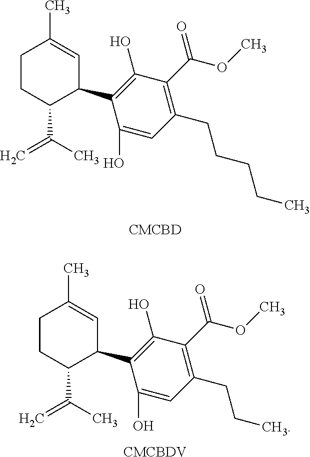

The compounds are (1′R,2′R)-methyl 2,6-dihydroxy-5′-methyl-4-pentyl-2′-(prop-1-en-2-yl)-1′,2′,3′,4′-tetrahydro-[1,1′-biphenyl]-3-carboxylate and (1′R,2′R)-methyl 2,6-dihydroxy-5′-methyl-4-propyl-2′-(prop-1-en-2-yl)-1 ‘,2’,3′,4′-tetrahydro-[1, 1′-biphenyl]-3-carboxylate and can be referred to as CMCBD and CMCBDV, respectively, throughout the application.

The present invention can produce purified CBD or CBDV in excellent yields.

### BRIEF DESCRIPTION OF THE DRAWINGS

FIG. 1 shows an illustration of a schematic process of the present invention.

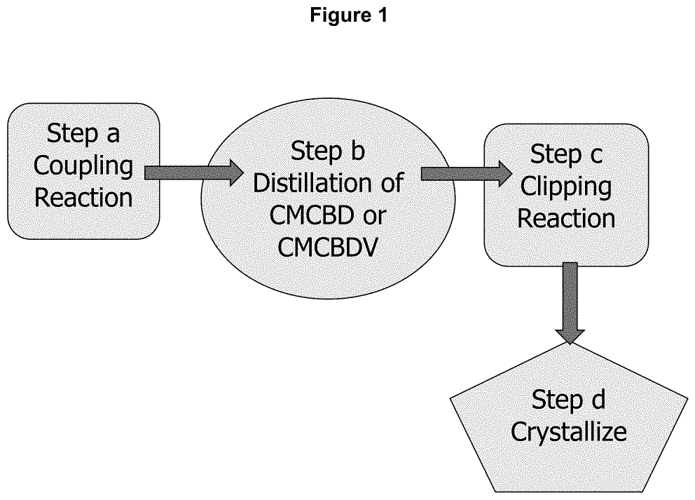

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification.

Unless otherwise defined, all technical and scientific terms used herein have the same meaning as commonly understood by one of ordinary skill in the art to which this invention pertains. In the case of conflict, the present document, including definitions will control.

“Around,” “about” or “approximately” shall generally mean within 10 percent, within 10 percent of a given value or range. Numerical quantities given are approximate, meaning that the term “around,” “about” or “approximately” can be inferred if not expressly stated.

“Cannabidiol” or “CBD” refers to a compound of the following structure:

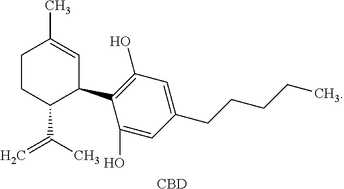

“Cannabidivarin” or “CBDV” refers to a compound of the following structure:

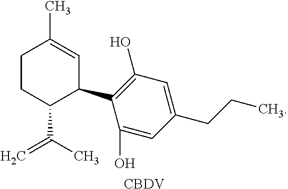

CMCBD refers to a compound of the following structure:

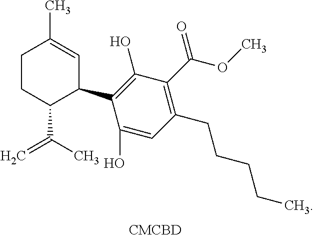 [SMILES: C=C(C)[C@@H]1CCC(C)=C[C@H]1c1c(O)cc(CCCCC)c(C(=O)OC)c1O]

CMCBDV refers to a compound of the following structure:

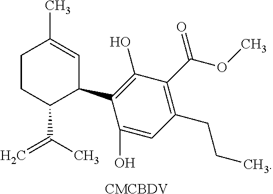

PMD refers to a compound of the following structure:

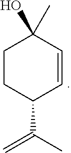

Carboxymethyl Olivetol (CMO) refers to a compound of the following structure:

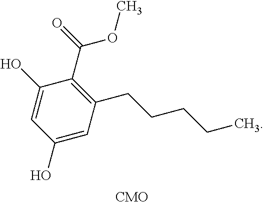 [SMILES: CCCCCc1cc(O)cc(O)c1C(=O)OC]

Carboxymethyl Divinarol (CMD) refers to a compound of the following structure:

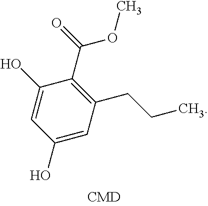

### Embodiments of the Invention

In one embodiment, the invention provides a method of manufacturing cannabidiol (CBD) comprising the following steps:

a) reacting p-mentha-2,8-diene-1-ol (PMD) with 6-carboxymethyl olivetol (CMO) and a catalyst (preferably, boron trifluoride (BF₃)-etherate) in the presence of an organic solvent to produce (1′R,2′R)-methyl 2,6-dihydroxy-5′-methyl-4-pentyl-2′-(prop-1-en-2-yl)-1′,2′,3′,4′-tetrahydro-[1,1′-biphenyl]-3-carboxylate (CMCBD);

b) distilling CMCBD to obtain a distilled CMCBD via thin film evaporation;

c) reacting the distilled CMCBD with water, methanol and sodium hydroxide to produce a CBD; and

d) purifying the CBD from step (c) to obtain a purified CBD.

In a preferred embodiment, the purifying of CBD in step (d) is performed via crystallization to obtain a crystallized CBD.

In another embodiment, the invention provides a method of manufacturing cannabidivarin (CBDV) comprising the following steps:

a) reacting p-mentha-2,8-diene-1-ol (PMD) with 6-carboxymethyl divinarol (CMD) and a catalyst (preferably, boron trifluoride (BF₃)-etherate) in the presence of an organic solvent to produce (1′R,2′R)-methyl 2,6-dihydroxy-5′-methyl-4-propyl-2′-(prop-1-en-2-yl)-1′,2′,3′,4′-tetrahydro-[1,1′-biphenyl]-3-carboxylate (CMCBDV);

b) distilling CMCBDV to obtain a distilled CMCBDV via thin film evaporation;

c) reacting the distilled CMCBDV with water, methanol and sodium hydroxide to produce a CBDV; and

d) purifying the CBDV from step (c) to obtain a purified CBDV.

In a preferred embodiment, the purifying of CBDV in step (d) is performed via crystallization to obtain a crystallized CBDV.

Step (a): “Coupling Reaction”

The reaction of step (a), a so-called “coupling reaction,” can be represented as follows:

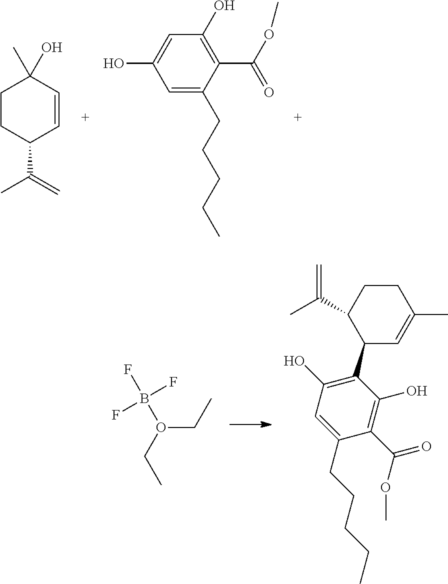

While BF₃-etherate is a preferred catalyst, other suitable catalysts can be used in the coupling reaction. Such suitable catalysts include, but are not limited to, scandium triflate, scandium chloride, ytterbium triflate, ytterbium chloride, tin chloride, titanium chloride aluminum trichloride, magnesium bromide as well as partial or fully substituted alkyl or alkoxy, phenyl or phenoxy derivatives of the same.

Preferably, the reaction results in at least 80% overall yield of CMCBD or CMCBDV. The main impurities, such as unreacted CMO, CMD and cis-CBD can be easily removed by methods known to those skilled in the art.

In a preferred embodiment, the molar ratio of PMD to CMO or CMD in step (a) is from 1:1 to 10:1.

Preferably, PMD is used in at least 30% molar excess compared to CMO or CMD.

In another preferred embodiment, step (a) is carried out at a temperature from 10° C. to 30° C.

In one embodiment, the organic solvent in step (a) is selected from dichloromethane, ethyl acetate, chloroform, methyl tert-butyl ether, cyclohexane, toluene, ethyl alcohol, methyl alcohol, isopropyl alcohol, n-butyl alcohol, tetrahydrofuran, dioxane, dimethylformamide, dimethyl sulfoxide, dimethylacetamide, methyl tert-butyl ether, cyclohexane, water and mixtures thereof.

In one embodiment, the invention also provides a compound of the following structure:

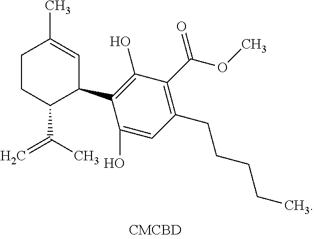 [SMILES: C=C(C)[C@@H]1CCC(C)=C[C@H]1c1c(O)cc(CCCCC)c(C(=O)OC)c1O]

This compound is (1′R,2′R)-methyl 2,6-dihydroxy-5′-methyl-4-pentyl-2′-(prop-1-en-2-yl)-1′,2′,3′,4′-tetrahydro-[1,1′-biphenyl]-3-carboxylate and is referred to as CMCBD throughout the application.

In another embodiment, the invention also provides a compound of the following structure:

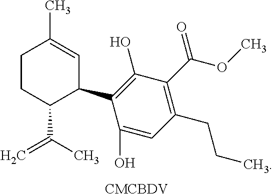

This compound is (1′R,2′R)-methyl 2,6-dihydroxy-5′-methyl-4-propyl-2′-(prop-1-en-2-yl)-1′,2′,3′,4′-tetrahydro-[1,1′-biphenyl]-3-carboxylate and is referred to as CMCBDV throughout the application.

Step (b): Distillation

In step (b), CMCBD or CMCBDV is distilled via thin film evaporation.

In one embodiment, the distilled CBD or CBDV obtained in step (b) has a purity of at least 90%.

In another embodiment, step (b) is carried out at a temperature from 70° C. to 170° C.

Briefly, the distillation process can be performed as follows:

Distillation may be accomplished via a three-stage system or a single stage system in multiple passes. In the first stage, the solvent is removed. For DCM, the jacket temperature is 140° C. with a vacuum of 100 torr and external condenser set to −15° C. The second stage is the deterpenylation stage wherein terpenes are removed. Its jacket temperature is 170° C. with a vacuum of 3-5 torr. Having been adequately desolvated and now without volatile terpenes, molecular distillation can then proceed via stage three with a jacket temperature of 170° C. and a vacuum of 100 mtorr. The most critical part of the distillation process is the removal of terpenes in stage two.

Step (c): “Clipping Reaction”

The reaction of step (c), a so-called “clipping reaction,” can be represented as follows:

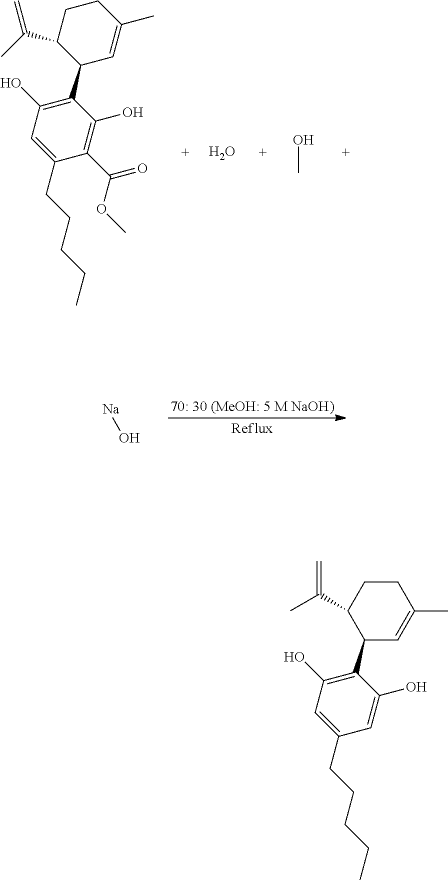

In one embodiment, the molar ratio of CMCBD or CMCBDV and water in step (c) is from 1:1 to 1:100.

In another embodiment, the molar ratio of methanol and sodium hydroxide in step (c) is from 1:1 to 1:100.

The clipping reaction can be carried out at a reflux temperature, including a temperature elevated by high pressure, of the solvent or solvent mixture for a duration of about 1, 2, 3, 4, 5, 6, 7, 8, 9, 10, 12, 16, 20, 24, 28, 30, 32, 36, 48 or about 120 hours; or any amount of time required to reach a desired endpoint (wherein the desired endpoint can be determined by for example, a percent conversion of starting material or an intermediate material). These values can define a range, such as about 10 to about 30 hours. In one embodiment, the reduction reaction can be carried out at reflux in a methanol-water mixture for a duration of about 16 hours to about 24 hours, or about 20 to about 28 hours.

The reflux temperature can be at 20° C., room temperature (RT), 30° C., 40° C., 50° C., 60° C., 65° C., 70° C., 75° C., 80° C., 85° C., 90° C., 95° C., 100° C., 110° C. or about 120° C. These values can be used to define a range, such as about 20° C. to about 100° C., or about RT to about 50° C., or about 60° C. to about 85° C., or about 72° C. to about 76° C.

Step (d): Purification (Preferably Via Crystallization)

After the “clipping reaction” of step (c), the obtained CBD or CBDV is purified, preferably via crystallization. Other methods to purify CBD or CBDV include chromatography (either normal or reverse phase), distillation, or sublimation.

Following decolorization, in one embodiment, a solid CBD or CBDV from step (c) is dissolved in a solvent at high temperature. As the solution cools, the solubility of the solute in the solvent will gradually become smaller. The resultant solution may be described as supersaturated, i.e. there is more solute dissolved in the solution than would be predicted by its solubility at that temperature. Crystallization can then be induced from this supersaturated solution by seeding with a small crystal seed mass of high purity and the resultant pure crystals removed by methods known to skilled artisans, including but not limited to vacuum filtration and centrifugal separators. The remaining solution, once the crystals have been filtered out, is known as the mother liquor, and will contain a portion of the original solute as well as any impurities that remained in solution as well.

Thus, in one embodiment of the invention it is possible to harvest second and third crops of crystals from the mother liquor.

In one embodiment, step (d) comprises using hexane and/or pentane and/or another suitable solvent as a crystallization solvent.

In one embodiment, CBD or CBDV is dissolved in 1:1 by mass hexane at 50° C. The solution is then cooled gradually to 20° C., at which time 1% w/w CBD or CBDV seed mass (with purity >95%) is pitched into the stirring closed container. The solution is then allowed to cool to −17° C. over 24 hours. Crystals harvested by this method are assessed for purity. If purity is insufficient to meet specification, they are crystallized once more by the same method. Once crystals meet purity specifications, they are crystallized one final time in pentane.

In another embodiment, the crystallized CBD or CBDV obtained in step (d) has a purity of at least 99%.

In a preferred embodiment, step (d) does not include chromatography purification, and therefore is a non-chromatographic process.

In one embodiment, the crystallized CBD or CBDV obtained in step (d) has the following crystal size distribution: between 250 μm and 1000 μm, with average size being 500 μm.

The invention also provides a crystallized CBD or CBDV manufactured by the method of manufacturing set forth herein.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

All claims, aspects and embodiments of the invention, and specific examples thereof, are intended to encompass equivalents thereof.

### EXAMPLES OF THE INVENTION

### Example 1

### Preparation of CMCBD (“Coupling Reaction”)

CMCBD was prepared as follows.

To 3980 mL dry DCM under N₂, CMO was added and dissolved. BF₃ was then added and allowed to stir for 20 min with the CMO. PMD in DCM (1:1) was then added via addition funnel rapidly. The reaction was monitored intermittently and ultimately quenched with an equivolume of saturated bicarbonate, and then analyzed via HPLC.

The reaction produced 781.65 g of CMCBD (MW 372.5) with a typical purity of 80%.

### Example 2

### Preparation of CBD (“Clipping Reaction”)

CBD was prepared as follows.

3 kg NaOH was added to 15 L cold water in the 100 L reactor while stirring and under N₂. An exotherm ensued and the temperature was noted. Once the temperature was below 65° C., 15 L MeOH was added. Then, 15 L of 8 kg CMCBD or CMCBDV in MeOH was added; reactor was topped off with MeOH to the 50 L mark. Then, heat was ramped up until 95° C. with condenser running. The reaction was checked periodically (quenched in phosphate buffer and analyzed by HPLC). The reaction took about 24 hours to complete. Once complete, MeOH was removed via distillation (vacuum) and the reaction allowed to cool. The removed MeOH was supplanted with the same volume (35 L) of 70:30 Hexane:H₂O solution. While gently stirring, CO₂ was bubbled until aqueous layer was neutral. The organic layer was removed and the aqueous layer was washed with 1 volume of hexane; dried; decolorized; solvent was removed and CBD.

### Example 3

### Crystallization of CBD

CBD was dissolved in 1:1 by mass hexane at 50° C. The solution was then cooled gradually to 20° C., at which time 1% w/w CBD seed mass (with purity >95%) was pitched into the stirring closed container. The solution was then allowed to cool to −17° C. over 24 hours. This procedure was repeated, as above, once more with hexane and a final time with pentane (dissolving in 35° C. pentane instead of 50° C.). Crystals were then filtered and solvent was removed by either N₂ blow down or vacuum or both.

### Example 4

### Preparation of CMCBDV (“Coupling Reaction”)

CMCBDV was prepared as follows.

To 4320 mL dry DCM under N₂, CMD was added and dissolved. BF₃ was then added and allowed to stir for 20 min with the CMD. PMD in DCM (1:1) was then added via addition funnel rapidly. The reaction was monitored intermittently and ultimately quenched with an equivolume of saturated bicarbonate, and then analyzed via HPLC.

The reaction produced 819.23 g of CMCBDV (MW 344.5) with a typical purity of 80%.

### Example 5

Preparation of CBDV (“Clipping Reaction”)

CBDV was prepared as follows.

200 g NaOH was added to 1 L cold water in a round bottom flask while stirring and under N₂. An exotherm ensued. Once the temperature was below 65° C., 2.3 L MeOH was added. Then, 500 mL L of 500 g CMCBDV in MeOH was added; the flask was topped off with MeOH to the 5 L mark. Then, the reaction was brought to 95° C. with condenser running and nitrogen gently bubbling. The reaction was checked periodically (quenched in phosphate buffer and analyzed by HPLC) for progress. The reaction took about 18 hours to complete. Once complete, MeOH was removed via vacuum distillation and the reaction allowed to cool. The removed MeOH was supplanted with the same volume (˜2 L) of 70:30 Hexane:H₂O solution. While gently stirring, CO₂ was bubbled until the aqueous layer was neutral. The organic layer was removed and the aqueous layer was washed with 1 volume of hexane; dried; solvent was removed and CBDV was crystallized. Yields typically encountered for this step are around 90%.

### Example 6

### Crystallization of CBDV

Crude CBDV from the above reaction was dissolved in 1:1 by mass hexane at 50° C. The solution was then cooled gradually to 20° C., at which time 1% w/w CBDV seed mass (with purity >95%) was pitched into the stirring closed container under gentle N₂ stream. The solution was then allowed to cool to −17° C. over 24 hours. This procedure was repeated, as above, once more with hexane and a final time with pentane (dissolving in 35° C. pentane instead of 50° C.). Crystals were then filtered and solvent was removed by either N₂ blow down or vacuum or both. Total mass recovery of the above crystallization scheme is about 65% with CBDV purity greater than 99.5%. Mother liquors from the above may be reworked, seeded, and crystallized again for additional crops; they may be added to different crude CBDV from the clipping reaction and further crystallized; or retained for chromatographic purification if the CBDV titer is sufficiently low as to not allow crystallization to occur.

What is claimed is:

1. A method of manufacturing cannabidiol (CBD) comprising the following steps:
a) reacting p-mentha-2,8-diene-1-ol (PMD) with 6-carboxymethyl olivetol (CMO) and a catalyst in the presence of an organic solvent to produce (1′R,2′R)-methyl 2,6-dihydroxy-5′-methyl-4-pentyl-2′-(prop-1-en-2-yl)-1′,2′,3′,4′-tetrahydro-[1,1′-biphenyl]-3-carboxylate (CMCBD);
b) distilling CMCBD to obtain a distilled CMCBD;
c) reacting the distilled CMCBD with water, methanol and sodium hydroxide to produce a CBD; and
d) purifying the CBD from step (c) to obtain a purified CBD.

2. The method of claim 1, wherein the catalyst comprises a compound selected from boron trifluoride (BF₃)-etherate, scandium triflate, scandium chloride, ytterbium triflate, ytterbium chloride, tin chloride, titanium chloride aluminum trichloride, magnesium bromide as well as partial or fully substituted alkyl, alkoxy, phenyl or phenoxy derivatives of the same.

3. The method of claim 1, wherein step (d) comprises crystallization and wherein the purified CBD is a crystallized CBD.

4. The method of claim 1, wherein the molar ratio of PMD to CMO in step (a) is from 1:1 to 10:1.

5. The method of claim 1, wherein PMD is at at least 30% molar excess compared to CMO.

6. The method of claim 1, wherein step (a) is carried out at a temperature from 10° C. to 30° C.

7. The method of claim 1, wherein the organic solvent in step (a) is selected from dichloromethane, ethyl acetate, chloroform, methyl tert-butyl ether, cyclohexane, toluene, ethyl alcohol, methyl alcohol, isopropyl alcohol, n-butyl alcohol, tetrahydrofuran, dioxane, dimethylformamide, dimethyl sulfoxide, dimethylacetamide, methyl tert-butyl ether, cyclohexane, water and mixtures thereof.

8. The method of claim 1, wherein the distilled CBD obtained in step (b) has a purity of at least 95%.

9. The method of claim 1, wherein step (b) is carried out at a temperature from 10° C. to 30° C.

10. The method of claim 1, wherein step (b) comprises thin film evaporation process.

11. The method of claim 1, wherein the molar ratio of CMCBD and water in step (c) is from 1:1 to 1:100.

12. The method of claim 1, wherein the molar ratio of methanol and sodium hydroxide in step (c) is from 1:1 to 1:100.

13. The method of claim 3, wherein step (d) comprises using hexane and/or pentane as a crystallization solvent.

14. The method of claim 3, wherein the crystallized CBD obtained in step (d) has a purity of at least 99%.

15. The method of claim 3, wherein the crystallized CBD obtained in step (d) has the following crystal size distribution: between 250 μm and 1000 μm, with average size of the crystal being 500 μm.

16. A crystallized CBD manufactured by the method of claim 3.

17. A compound of the following structure:

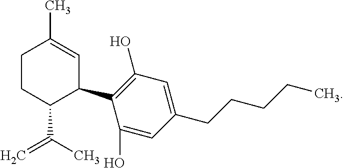 [SMILES: C=C(C)[C@@H]1CCC(C)=C[C@H]1c1c(O)cc(CCCCC)cc1O]

18. A method of manufacturing cannabidivarin (CBDV) comprising the following steps:
a) reacting p-mentha-2,8-diene-1-ol (PMD) with 6-carboxymethyl divinarol (CMD) and a catalyst in the presence of an organic solvent to produce (1′R,2′R)-methyl 2,6-dihydroxy-5′-methyl-4-propyl-2′-(prop-1-en-2-yl)-1′,2′,3′,4′-tetrahydro-[1,1′-biphenyl]-3-carboxylate (CMCBDV);
b) distilling CMCBDV to obtain a distilled CMCBDV;
c) reacting the distilled CMCBDV with water, methanol and sodium hydroxide to produce a CBDV; and
d) purifying the CBDV from step (c) to obtain a purified CBDV.

19. The method of claim 18, wherein step (d) comprises crystallization and wherein the purified CBDV is a crystallized CBDV.

20. A crystallized CBDV manufactured by the method of claim 19.
